(19) United States
(12) Patent Application Publication
(10) Pub. No.: US 2011/0251126 A1
(43) Pub. Date: Oct. 13, 2011

(54) INTESTINAL TREATMENT

(76) Inventors: Christian Elling, Hoersholm (DK); Thue Schwartz, Hoersholm (DK); Niels Ole Jensen, Hoersholm (DK); Lars-Ole Gerlach, Hoersholm (DK)

(21) Appl. No.: 13/062,932

(22) PCT Filed: Sep. 11, 2009
(86) PCT No.: PCT/EP2009/006604

§ 371 Date: Jun. 6, 2011

(30) Foreign Application Priority Data
Sep. 18, 2008 (GB) 0817067.2

(51) Int. Cl. A61K 38/17; A61P 1/12; A61P 29/00; A61P 1/00

(52) U.S. Cl. 514/13.2; 514/21.3

(57) ABSTRACT

Y4 receptor agonists which are selective for the Y4 receptor over the Y1 and Y2 receptors, are useful in the prevention and/or treatment of damage to bowel function caused by radiation therapy, radiation exposure, cytotoxic chemotherapy, inflammation, or ischemia-reperfusion of intestinal mucosa.

This invention relates to the use of a Y4 receptor agonist which is selective for the Y4 receptor relative to the Y1 and Y2 receptors, in the prevention and/or treatment of damage to bowel function caused by radiation therapy, radiation exposure, cytotoxic chemotherapy, inflammation or ischemia-reperfusion of intestinal mucosa.

### BACKGROUND TO THE INVENTION

The PP-fold family of peptides—NPY (Neuropeptide Y) (human sequence—SEQ ID. No:1), PYY (Peptide YY) (human sequence—SEQ ID. No:2), and PP (Pancreatic Polypeptide) (human sequence—SEQ ID. No:3), are naturally secreted homologous, 36 amino acid, C-terminally amidated peptides, which are characterized by a common three-dimensional, structure—the PP-fold—which is surprisingly stable even in dilute aqueous solution and is important for the receptor recognition of the peptides.

The PP-fold structure common to NPY, PYY and PP consists of 1) an N-terminal polyproline-like helix (corresponding to residues 1 through 8 with Pro2, Pro5, and Pro8) followed by 2) a type I beta-turn region (corresponding to residues 9 through 12) followed by 3) an amphiphilic alpha-helix (residues 13-30) which lies anti-parallel to the polyproline helix with an angle of about 152 degrees between the helical axes, and 4) a C-terminal hexapeptide (residues 31-36). The folded structure is stabilized through hydrophobic interactions between side chains of the amphiphilic alpha-helix which are closely interdigitating with the three hydrophobic proline residues (Schwartz et al 1990). Besides key residues in the receptor recognizing C-terminal hexapeptide it is the core hydrophobic residues, which stabilize the PP-fold structure, which are conserved across the family of PP-fold peptides.

NPY is a very wide-spread neuropeptide with multiple actions in various parts of both the central and peripheral nervous system acting through a number of different receptor subtypes in man: Y1, Y2, Y4 and Y5. The main NPY receptors are the Y1 receptor, which generally is the post-synaptic receptor conveying the “action” of the NPY neurones and the Y2 receptor which generally is a pre-synaptic, inhibitory receptor. This is also the case in the hypothalamus, where NPY neurones—which also express the melanocortin receptor antagonist/inverse agonist AgRP (agouti related peptide)—act as the primary “sensory” neurones in the stimulatory branch of the arcuate nucleus. Thus, in this the “sensor nucleus” for the control of appetite and energy expenditure, the NPY/AgRP neurones together with the inhibitory POMC/CART neurones monitor the hormonal and nutritional status of the body as these neurones are the target for both the long-term regulators such as leptin and insulin and short term regulators such as ghrelin and PYY (see below). The stimulatory NPY/AgRP neurones project for example to the paraventricular nucleus—also of the hypothalamus—where its postsynaptic target receptors are believed to be Y1 and Y5 receptors. NPY is the most potent compound known in respect of increasing food intake, as rodents upon intracerebroventricular (ICV) injection of NPY will eat until they literally burst. AgRP from the NPY/AgRP neurones acts as an antagonist mainly on melanocortin receptors type 4 (MC-4) and block the action of POMC derived peptides—mainly aMSH—on this receptor. Since the MC4 receptor signal acts as an inhibitor of food intake, the action of AgRP is—just like the NPY action—a stimulatory signal for food intake (i.e. an inhibition of an inhibition). On the NPY/AGRP neurons are found inhibitory—pre-synaptic—Y2 receptors, which are the target both of locally released NPY as well as a target for the gut hormone PYY—another PP-fold peptide.

PYY is released during a meal—in proportion to the calorie content of the meal—from entero-endocrine cells in the distal small intestine and the colon, to act both in the periphery on GI-tract functions and centrally as a satiety signal. Peripherally, PYY is believed to function as an inhibitor—an “illeal break”—on for example upper GI-tract motility, gastric acid and exocrine pancreatic secretion. Centrally, PYY is believed to act mainly on the presynaptic, inhibitory Y2 receptors on the NPY/AgRP neurones in the arcuate nucleus, which it is believed get access to from the blood (Batterham et al. 2002 Nature 418: 650-4). The peptide is released as PYY1-36, but a fraction—approximately 50%—circulates as PYY3-36 which is a product of degradation by dipeptidylpeptidase-IV an enzyme which removes a dipeptide from the N-terminus of a peptide provided that a Pro or Ala is found in position two as in all three PP-fold peptides—PP, PYY and NPY (Eberlein et al. 1989 Peptides 10: 797-803). Thus PYY in the circulation is a mixture of PYY1-36, which acts on both Y1 and Y2 receptors (as well as Y4 and Y5 with various affinities), and PYY3-36, a highly potent Y2 agonist with lower affinities for the Y1, Y4 and Y5 receptors than for the Y2 receptor. In the Y-receptor potency assays described below, PYY3-36 is more than 10,000 fold more potent towards the Y2 receptor than towards the Y4 receptor.

PP is a hormone, which is released from endocrine cells in the pancreatic islets, almost exclusively governed by vagal cholinergic stimuli elicited by especially food intake (Schwartz 1983 Gastroenterology 85:1411-25). PP has various effects on the gastrointestinal tract, but none of these are observed in isolated cells and organs, and all appear to be dependent on an intact vagal nerve supply (Schwartz1983 Gastroenterology 85:1411-25). In accordance with this, the PP receptors, which are called Y4 receptors, are located in the brain stem with a strong expression in vagal motor neurones—activation of which results in the peripheral effects of PP—and in the nucleus tractus solitarirus (NTS)—activation of which results in the effects of PP as a satiety hormone (Whitecomb et al. 1990 Am. J. Physiol. 259: G687-91, Larsen & Kristensen 1997 Brain Res. Mol. Brain Res 48: 1-6). It should be noted that PP from the blood has access to this area of the brain since the blood brain barrier is “leaky” in this area where various hormones from the periphery are sensed. Recently it has been argued that part of the effect of PP on food intake is mediated through an action on neurones—especially the POMC/CART neurones in the arcuate nucleus (Batterham et al. 2004 Abstract 3.3 International NPY Symposium in Coimbra, Portugal). PP acts through Y4 receptors for which it has a subnanomolar affinity as opposed to PYY and NPY which have nanomolar affinity for this receptor (Michel at al. 1998 Pharmacol. Rev. 50: 143-150). PP also has an appreciable affinity for the Y5 receptor, but it is not likely of physiological importance in relation to circulating PP due to both lack of access to the cells in the CNS where this receptor especially is expressed and due to the relatively low affinity for PP.

### PP-Fold Peptide Receptors

There are four well established types of PP-fold peptide receptors in man: Y1, Y2, Y4, and Y5 which all recognize NPY1-36 and PYY1-36 within a 100 fold affinity range. At one time, a Y3 receptor type, which might prefer NPY over PYY, was suggested, but today this is not accepted as a real receptor subtype (Michel at al. 1998 Pharmacol. Rev. 50: 143-150). A Y6 receptor subtype has been cloned, which in man is expressed in a truncated form lacking TM-VII as well as the receptor tail and consequently at least on its own does not appear to form a functional receptor molecule.

Y1 receptors—affinity studies suggest Y1 binds NPY and PYY equally well and basically not PP. Affinity for Y1 is dependent on the identities of both end sequences of the PP-fold molecule (NPY/PYY)—for example residues Tyr1 and Pro2 are essential—and it is dependent on the peptide ends being presented in just the right way. In the C-terminal end, where the side-chains of several of the residues are essential, the Y1 receptor—like the Y5 and Y4 receptor but not the Y2 receptor—tolerates certain substitutions in position 34 (normally a Gln)—such as Pro (Fuhlendorff of al. 1990 J. Biol. Chem. 265: 11706-12, Schwartz et al. 1990 Annals NY Acad. Sci. 61: 35-47). Some structure-function studies concerning the requirements of the Y1 and Y2 receptors have been reported (Beck-Sickinger et al. 1994 Eur. J. Biochem. 225: 947-58; Beck-Sickinger and Jung 1995 Biopolymers 37: 123-42; Soil et al. 2001 Eur. J. Biochem. 268: 2828-37).

Y2 receptors—affinity studies suggest Y2 binds NPY and PYY equally well and basically not PP. The receptor requires especially the C-terminal end of the PP-fold peptide (NPY/PYY). Thus, long C-terminal fragments—down to for example NPY13-36 (the whole alpha helix plus the C-terminal hexapeptide)—are recognized with relatively high affinity, i.e. to within ten-fold of the affinity of the full-length peptide (Sheikh et al. 1989 FEBS Lett. 245: 209-14, Sheikh et al. 1989 J. Biol. Chem. 264: 6648-54). Therefore various N-terminal deletions, which eliminate the binding to the Y1 receptor, still preserve some degree of binding to the Y2 receptor. However, the affinity of the C-terminal fragments is reduced approximately 10 fold as compared to NPY/PYY for even relatively long fragments. The Gln residue in position 34 of NPY and PYY is highly important for the ligand recognition of the Y2 receptor (Schwartz et al. 1990 Annals NY Acad. Sci. 611: 35-47).

Y4 receptors—affinity studies suggest that Y4 binds PP with subnanomolar affinity corresponding to the concentrations found in plasma whereas NPY and PYY are recognized with much lower affinity. Such studies suggest the Y4 receptor is highly dependent on the C-terminal end of the PP-fold peptides, and that relatively short N-terminal deletions impairs the affinities of the ligands. Some structure activity studies concerning the Y4 receptor have been reported (Gehlert et al. 1996 Mol. Pharmacol. 50: 112-18; Walker et al. 1997 Peptides 18: 609-12).

Y5 receptors—affinity studies suggest that Y5 binds NPY and PYY equally well, and also binds PP with lower affinity, which however is below the normal circulating levels of this hormone. PYY3-36 is also recognized well by the Y5 receptor, however this receptor is to a large degree expressed in the CNS where such peptide cannot get access to the receptor readily when administered in the periphery.

From the above summaries, it is clear that the natural PP-fold Y-receptor peptide agonists have different selectivity profiles for the various Y-receptors. International patent applications WO 2005/089786 and WO 2007/038942 show that modified PP-fold peptides can be created which have selectivity profiles favouring the Y4 receptor over the Y1 and Y2 receptors. For example, following the disclosures of those publications, Y-receptor peptide agonists have been created which have at least 200 fold greater potency at the Y4 receptor than at the Y1 receptor, and at least 1000 fold greater potency at the Y4 receptor than at the Y2 receptor. In this connection, it should be noted that the binding affinity of an agent to a particular receptor is not usually predictive of the potency of the agent at that receptor, nor does it predict the functionality of the agent at that receptor, ie whether it has agonist, antagonist, partial agonist or other functionality.

The affinity of a peptide to a specific receptor is given for example as an IC₅₀ value or a Kᵢ or K_{d} value, which in a specific, non-limiting example is determined in an assay, such as a competition binding assay. The IC50 value corresponds to the concentration of the peptide which displaces a—for the given receptor relevant—radioactive ligand used in an amount far less than the Kd for that radioactive ligand to 50%.

In vitro potency of a compound is defined in terms of EC₅₀ values, i.e. the concentration that leads to 50% of the maximally achievable effect as determined in a for the given receptor relevant signalling assay, such as the potency assay described herein.

### Damage to Mucosal Function

The mucosa is the innermost layer of the gastrointestinal tract that is surrounding the lumen, or space within the tube. This layer comes in direct contact with the food (or bolus), and is responsible for absorption and, important processes in digestion.

The mucosae are highly specialized in each organ of the gastrointestinal tract, facing a low pH in the stomach, absorbing a multitude of different substances in the small intestine (upper bowel), and also absorbing specific quantities of water in the large intestine (lower bowel).

Chemotherapy has contributed for improving survival of patients with malignant disorders. Although peripheral blood stem cell rescue can improve the dose-limitations of anti-cancer drug treatment, bowel mucosal cell toxicity can be a dose-limiting toxicity for cancer treatment. Radiation and high doses of anti-cancer drugs cause severe mucostitis, which not only distresses patients with pain and diarrhea but also increases the risk of infection. Mucositis is the painful inflammation and ulceration of the mucous membranes lining the digestive tract, usually as an adverse effect of cytotoxic chemotherapy and radiotherapy treatment for cancer.

Acute radiation injury to the small intestine has been well documented in animal models after abdominal radiation exposure. It is characterized by cell loss in the progenitor cell compartment (impaired epithelial renewal, villus atrophy), microvascular endothelial cell death (local ischemia), and mucosal inflammation (loss of barrier properties, epithelial atypia/mucosal ulceration).

Acute radiation enteritis or radiation induced intestinal dysfunction occurs in 75% of patients undergoing radiation therapy, typically occurring in the second or third week of therapy. The symptoms can be characterized by abdominal cramping and diarrhea—a serious and feared side effect that may result in insufficient cancer treatment and/or increased overall treatment time due to lower daily dosing or even cessation of therapy as well as reduced quality of life and can even result in death. In 5-15% of patients, the condition becomes chronic. In addition to discomfort, these side effects decrease the therapeutic benefit from radiation treatment by increasing the overall treatment time. (MacNaughton, W. K. Aliment. Pharmacol. Ther. 2000, 14, 523-528; Nguyen, N. P.; Antoine, J. E.; Dutta, S.; Karlsson, U.; Sallah, S. Cancer 2002, 95, 1151-1163; Gwede, C. K. Sem. Nursing Oncol. 2003, 19, 6-10.)

Exposure to radiation can occur in several other ways, including exposure to normal background levels of radiation (such as cosmic rays or radiation due to naturally occurring isotopes present in the earth) or elevated environmental radiation (including occupational exposure of persons in medical facilities or nuclear power plants as well as exposure to X-rays during medical diagnosis). Another potential source of exposure to certain types of radiation is the accidental or intentional release of radioactive materials, for example of an accident or as a result of terrorist activity, e.g., as the result of a nuclear weapon such as a so-called “dirty bomb” (an explosive device intended to spread radioactive materials to contaminate an area).

Inflammation of the bowel, for example due to ulcerative colitis or Crohn's disease; and ischemia and subsequent reperfusion of intestinal mucosa also result in damage to the proper functioning of the bowel.

Diarrhea is main symptom of damage to bowel function caused by radiation therapy, radiation exposure, cytotoxic chemotherapy, inflammation or ischemia-reperfusion of intestinal mucosa.

### Y Receptor Peptides & Mucosa

### Intestinal Hypersecretion

In vivo studies have shown that PYY or NPY infusion to healthy human subjects attenuate intestinal hypersecretion prestimulated by either prostaglandin E2 or vasoactive intestinal polypeptide (VIP) (Holzer-Petsche U, Petritsch W, Hinterleitner T, Eherer A, Sperk G, Krejs G J. Gastroenterology 1991; 101:325-30 and Playford R J, Domin J, Parmar K B, Tatemoto K, Bloom S R, Calam J. Lancet 1990; 335:1555-7). Recent studies have shown that PYY, PYY(3-36), NPY and PP are anti-secretory and these peptides stimulate the same repertoire of Y receptors (Y1, Y2 and Y4) in human and mouse tissue (Cox H M, Pollock E L, Tough I R, Herzog H. Peptides 2001; 22:445-52; Cox H M, Tough I R. Br J Pharmacol 2002; 135:1505-12; Hyland N P, Sjöberg F, Tough I R, Herzog H, Cox H M. Br J Pharmacol 2003; 139:863-71). This has been demonstrated primarily by functional studies utilizing isolated tissues from genetically modified mice lacking either a single Y receptor (Y1, Y2 or Y4) or single peptide KO (“knock out”) tissues. For example, PP mediates an anti-secretory effect through the Y4 receptor solely located to the epithelium, in human tissue and mouse colon.

EP 1902730 relates to the use of NPY, the natural Y2 receptor-selective agonist in the treatment of hypersecretory diarrhea.

International patent applications WO 2005/089786 and WO 2007/038942 which, as mentioned above, relate to PP-fold peptides which are selective agonists of the Y4 receptor relative to the Y1 and Y2 receptors, also refer to the anti-secretory effects of those Y4-selective peptides, and their consequential utility in treatment of hypersecretory diarrhea.

The mucosal loss and damage to mucosal function caused by radiation therapy, radiation exposure, cytotoxic chemotherapy, inflammation or ischemia-reperfusion of intestinal mucosa also results in diarrhea, but the underlying cause is not mucosal hypersecretion. The antisecretory effects of PP-fold peptide agonists of the Y-receptors is not predictive of the ability of those agents to treat mucosal cell loss and damage to mucosal function due to radiation therapy, radiation exposure, cytotoxic chemotherapy, inflammation or ischemia-reperfusion.

However, International patent application WO 03/105763 demonstrates the ability of PYY[3-36], a Y2 receptor specific agonist (see above), to reduce colonic damage in an animal model for inflammatory bowel disease. This suggests that stimulation of the Y2 receptor may be a strategy for protection against mucosal loss and loss of mucosal function where the underlying cause is reduction of the normal cellular restorative function in the bowel.

### BRIEF SUMMARY OF THE INVENTION

This invention is based on the finding that Y4 receptor agonist which is selective for the Y4 receptor relative to the Y1 and Y2 receptors, has a protective effect against loss of intestinal (i.e. bowel) function caused by radiation therapy, radiation exposure, cytotoxic chemotherapy, inflammation or ischemia-reperfusion of intestinal mucosa.

### DETAILED DESCRIPTION OF THE INVENTION

In one aspect, the invention provides the use of a Y4 receptor agonist which has at least 50 fold greater potency at the Y4 receptor than at the Y1 receptor, and at least 1000 fold greater potency at the Y4 receptor than at the Y2 receptor, in the prevention and/or treatment of, or in the manufacture of a composition for treatment of, damage to bowel function caused by radiation therapy, radiation exposure, cytotoxic chemotherapy, inflammation or ischemia-reperfusion of intestinal mucosa.

Damage to bowel function may be caused by inflammatory bowel disease, for example ulcerative colitis or Crohn disease.

The Y4 receptor agonist used according to the invention is one which selectively stimulates the Y4 receptor relative to the Y1 and Y2 receptors. For present purposes a suitable selective Y4 agonist has at least 50 fold, preferably 100 fold, and more preferably 200 fold greater potency at the Y4 receptor than at the Y1 receptor, and at least 1000 fold greater potency at the Y4 receptor than at the Y2 receptor. Assays for determination of agonist potency at the Y receptors are known, but the potency assay described in the Examples section below is the intended assay for determination of whther a given Y4 receptor agonist meets the selectivity criteria specified herein.

Preferably, the Y4 receptor agonist used according to the invention has at least 200 fold greater potency at the Y4 receptor than at the Y1 receptor, and at least 1000 fold greater potency at the Y4 receptor than at the Y2 receptor

The invention is not restricted to use of any specific selective Y4 receptor agonist. Any such agonist meeting the selectivity definition herein may be used. International patent application WO 2005/089786 (the disclosures of which are hereby incorporated herein by reference) gives principles and instructions concerning the design of selective Y4 receptor agonists which are peptidic in character, and any peptide agonist made following those principles and instructions which meets the potency definition of Y4 receptor selectivity herein may be used.

In accordance with the disclosures of WO 2005/089786, modification of the natural hPP Y4 receptor agonist can yield Y4 receptor agonists which meet the selectivity definition herein. For the following discussion:
The notation hPP used herein refers to the hPP sequence (SEQ ID No:3). Thus the designation “[Ala30]hPP” specifies the human PP sequence (SEQ ID No: 3) but with alanine substituted for leucine at position 30 thereof.
The notation PP₂₋₃₆ used herein refers to the PP sequence (SEQ ID No:3) but with the first N-terminal amino acid (Ala) deleted. However, the position numbering of PP₂₋₃₆ is by reference to the full length PP (SEQ ID No:3). Thus, the designation “[Ala30]PP₂₋₃₆” specifies the human PP sequence SEQ ID No:3, but with Ala1 deleted, and alanine substituted for leucine at position 30 of SEQ ID No:3.
The notation PP₃₋₃₆ used herein refers to the PP sequence (SEQ ID No:3) but with the first two N-terminal amino acid residues (Ala and Pro) deleted. However, the position numbering of PP₃₋₃₆ is by reference to the full length PP (SEQ ID No:3). Thus, the designation “[Ala30]PP₃₋₃₆” specifies the human PP sequence SEQ ID No:3, but with Ala1 and Pro2 deleted, and alanine substituted for leucine at position 30 of SEQ ID No:3.

In this specification, reference is made to amino acids by their common names or abbreviations, such as valine (Val), leucine (Leu), isoleucine (Ile), methionine (Met), phenylalanine (Phe), asparagine (Asn), glutamic acid (Glu), glutamine (Gln), histidine (His), lysine (Lys), arginine (Arg), aspartic acid (Asp), glycine (Gly), alanine (Ala), serine (Ser), threonine (Thr), tyrosine (Tyr), tryptophane (Trp), cysteine (Cys) and proline (Pro). When referred to by its common name or abbreviation, without specifying its steroisomeric form, the amino acid in question is to be understood as the L-form.

Selective Y4 receptor agonists for use in accordance with the invention include those of SEQ ID Nos: 3-35 herein, and their conservatively subtituted analogues. The term “conservative substitution” as used herein denotes that one or more amino acids is replaced by another, biologically similar residue. Examples include substitution of amino acid residues with similar characteristics, e.g. small amino acids, acidic amino acids, polar amino acids, basic amino acids, hydrophobic amino acids and aromatic amino acids. Non-limiting examples of conservative amino acid substitutions suitable for use in the present invention include those in the following Table and analogous substitutions of the original residue by non-natural alpha amino acids which have similar characteristics. For example, as discussed below, Met residues may be substituted with norleucine (Nle) which is a bioisostere for Met, but which—as opposed to Met—is not readily oxidised. Another example of a conservative substitution with a residue normally not found in endogenous, mammalian peptides and proteins would be the conservative substitution of Arg or Lys with for example, ornithine, canavanine, aminoethylcysteine or other basic amino acid. For further information concerning phenotypically silent substitutions in peptides and proteins, see, for example, Bowie et. al. Science 247, 1306-1310, 1990.

| Original residue | Conservative substitution |
|---|---|
| Ala | Gly |
| Arg | Lys |
| Asn | Gln, His, Thr |
| Asp | Glu |
| Gln | Asn, His |
| Glu | Asp |
| His | Asn, Gln |
| Ile | Leu, Val |
| Leu | Ile, Val |
| Lys | Arg |
| Met | Leu, Ile |
| Phe | Tyr, Trp, His |
| Ser | Thr, Asn |
| Thr | Ser, Asn, Gln |
| Trp | Tyr, Phe, His |
| Tyr | Trp, Phe, His |
| Val | Ile, Leu |

Conservatively substituted analogues of the invention may have, for example, up to 10 conservative substitutions, or in another embodiment up to 5, or in yet another embodiment 3 or fewer. Preferably conservatively substituted analogues of SEQ ID Nos: 3-35 maintain the 5 N- and C-terminal amino acids of those sequences.

In the hPP sequence Asp10 is particularly prone to cyclisation in solution to form a cyclic imidate which ring opens to form mixtures of the α and β-aspartate with concomitant scrambling of stereochemistry. Conservative substitution of Asp at this position, ie by a residue which preserves the electrostatic potential distribution within the peptide, is therefore beneficial, since the overall stability and solubility of the peptide is thereby preserved. Glu is a suitable replacement for Asp. In position 10 it does not undergo analogous cyclisation/ring opening to form y-Glu it has the beneficial effect of improving the bulk and the solution stability of the peptide as a pharmaceutical agent compared to its Asp10 counterparts. Improved solution stability leads to increased synthetic yields and reduces the requirement for troublesome, costly and waste producing purification of the desired product from the closely related 13-Asp impurity.

The Met17 and Met30 residues in the normal hPP sequence can potentially undergo oxidation upon storage in solution. Met30 may therefore be conservatively substituted with a residue that is not prone to this alteration, such as Thr, Asn, Glu or Nle. Met17 may be conservatively replaced by Leu or Nle which prevents oxidation at this position and preserves the aliphatic side chain structure.

The existence of the Ala1-Pro2 motif in the normal hPP sequence confers upon that peptide an inherent instability towards the β-ketopiperazine degradation pathway in which the terminal amino function can ‘bite back’ via a 6 membered transition state that is stabilized by the turn inducing Pro, and undergo an intramolecular transamidation at the site of the proline carboxamide function leading to the formation of 3-ketopiperazine and hPP3-36. This pathway leads to degradation products formed on storage of the lyophilates, and significant degradation in solutions of peptides containing the Ala1-Pro2 sequence. Thus in preferred Y4 selective agonists for use in the invention this is prevented by removal of Ala1 from the PP sequence. This has the beneficial effect of improving the stability of these peptides both in solution and as lyophilates and therefore improving their properties as pharmaceuticals. Furthermore, the removal of Ala1 from the PP sequence reduce potency of the peptide to the Y1 receptor thus increasing the selectivity between the Y1 and Y4 receptor.

For the above reasons, preferred Y4 selective agonists for use in the present invention have the hPP, hPP₂₋₃₆ or hPP₃₋₃₆ sequence, but with the purposive conservative modifications at one or more of positions 10, 17 and 30 discussed above.

Specific selective Y4 receptor agonists referred to in WO 2005/089786 and WO 2007/038942 which are suitable for use according to the present invention include:
hPP (SEQ ID No: 3), hPP₂₋₃₆ (SEQ ID No: 4) and hPP₃₋₃₆ (SEQ ID No: 5)
[Ala30]hPP₂₋₃₆ (SEQ ID No: 6) and [Ala30]hPP (SEQ ID No: 7) and [Ala30]hPP₃₋₃₆ (SEQ ID No: 8)

[Thr30]hPP₂₋₃₆ (SEQ ID No: 9) and [Thr30]hPP (SEQ ID No: 10) and [Thr30]hPP₃₋₃₆ (SEQ ID No: 11)

[Asn30]hPP₂₋₃₆ (SEQ ID No: 12) and [Asn30]hPP (SEQ ID No: 13) and [Asn30]hPP₃₋₃₆ (SEQ ID No: 14)

[Gln30]hPP₂₋₃₆ (SEQ ID No: 15) and [Gln30]hPP (SEQ ID No: 16) and [Gln30]hPP₃₋₃₆ (SEQ ID No: 17)

[Glu10]hPP₂₋₃₆ (SEQ ID No: 18) and [Glu10]hPP (SEQ ID No: 19) and [Glu10]hPP₃₋₃₆ (SEQ ID No: 20)

[Glu10,Leu17,Thr30]hPP₂₋₃₆ (SEQ ID No: 21) and [Glu10,Leu17,Thr30]hPP (SEQ ID No: 22) and [Glu10,Leu17,Thr30]hPP₃₋₃₆ (SEQ ID No: 23)

[Nle17,Nle30]hPP₂₋₃₆ (SEQ ID No: 24) and [Nle17,Nle30]hPP (SEQ ID No: 25) and [Nle17,Nle30]hPP₃₋₃₆ (SEQ ID No: 26)

[Glu10,Nle17,Nle30]hPP₂₋₃₆ (SEQ ID No: 27) and [Glu10,Nle17,Nle30]hPP (SEQ ID No: 28) and [Glu10,Nle17,Nle30]hPP₃₋₃₆ (SEQ ID No: 29)

[Leu17; Thr30]hPP₂₋₃₆(SEQ ID No: 30) and [Leu17; Thr30]hPP (SEQ ID No: 31) and [Leu17; Thr30]hPP₃₋₃₆(SEQ ID No: 32)

[Leu17; Ser30]hPP₂₋₃₆(SEQ ID No: 33) and [Leu17; Ser30]hPP (SEQ ID No: 34) and [Leu17; Ser30]hPP₃₋₃₆(SEQ ID No: 35)

A currently preferred selective Y4 receptor agonist for use in accordance with the invention is PP2-36 (SEQ ID No: 4).

As is known in the art of peptide therapeutic compounds, various modifications to the basic peptide structure may be made with the purpose of modifying their stability or in vivo properties. Examples of such modifications, which may be present in the Y4 selective agonists for use in the invention, including those of SEQ ID Nos: 3-35 above, conservative substitution as discussed above, and those discussed below:

### N-Acylated Analogues

Y4 selective agonists with which the invention is concerned may be acylated at their N-terminus to confer resistance to aminopeptidases. For example, acylation may be with a carbon chain having from 2 to 24 carbon atoms, and N-terminal acetylation is a particular example.

Analogues with Covalently Bound Functional Motifs

Various modifications may be made to the Y4 selective agonists with which the invention is concerned, for the purpose of improving their pharmacokinetics, pharmacodynamics and metabolic properties. Such modifications may involve linking the agonist to functional groupings (also known as motifs) known per se in the art of peptidic or proteinaceous pharmaceuticals. Three particular modifications of particular benefit in the case of the agonists with which the invention is concerned, are linkage with serum albumin binding motifs, or glycosaminoglycan (GAG) binding motifs, or PEGylation.

Serum-Albumin Binding Motifs

Serum albumin binding motifs are typically lipophilic groups, incorporated to enable a prolonged residence in the body upon administration or for other reasons, which may be coupled in various known ways to peptidic or proteinaceous molecules, for example i) via a covalent linkage to e.g. a functional group present on a side-chain amino acid residue, ii) via a functional group inserted in the peptide or in a suitable derivatized peptide, iii) as an integrated part of the peptide. For example, WO 96/29344 (Novo Nordisk A/S) and P. Kurtzhals et al. 1995 Biochemical J. 312: 725-31, describe a number of suitable lipophilic modifications which can be employed in the case of the agonists with which this invention is concerned.

Suitable lipophilic groups include optionally substituted, saturated or unsaturated, straight or branched hydrocarbon groups of from 10 to 24 carbon atoms. Such groups may form, or may form part of, a side chain to the backbone of the agonist, for example by ether, thioether, amino, ester or amide linkage to a side chain of an amino acid residue in the backbone, or to a backbone carbon or a branch from a backbone carbon of a non-peptidic linker radical in the backbone of a PP-fold mimic agonist. The chemistry strategy for attachment of the lipophilic group is not critical, but the following side chains including lipophilic groups are examples which can be linked to a backbone carbon of the agonist, or suitable branch therefrom:
CH₃(CH₂)ₙCH(COOH)NH—CO(CH₂)₂CONH— wherein n is an integer from 9 to 15,
CH₃(CH₂)ᵣCO—NHCH(COOH)(CH₂)₂CONH— wherein r is an integer form 9 to 15,
CH₃(CH₂)ₛCO—NHCH((CH₂)₂COOH)CONH— wherein s is an integer from 9 to 15,
CH₃(CH₂)ₘCONH—, wherein m is an integer from 8 to 18,
—NHCOCH((CH₂)₂COOH)NH—CO(CH₂)ₚCH₃, wherein p is an integer from 10 to 16,
—NHCO(CH₂)₂CH(COOH)NH—CO(CH₂)_{q}CH₃, wherein q is an integer from 10 to 16,
CH₃(CH₂)ₙCH(COOH)NHCO—, wherein n is an integer from 9 to 15,
CH₃(CH₂)ₚNHCO—, wherein p is an integer from 10 to 18,
—CONHCH(COOH)(CH₂)₄NH—CO(CH₂)ₘCH₃, wherein m is an integer from 8 to 18,
—CONHCH(COOH)(CH₂)₄NH—COCH((CH₂)₂COOH)NH—CO(CH₂)ₚCH₃, wherein p is an integer from 10 to 16,
—CONHCH(COOH)(CH₂)₄NH—CO(CH₂)₂CH(COOH)NH—CO(CH₂)_{q}CH₃, wherein q is an integer from 10 to 16, and
a partly or completely hydrogenated cyclopentanophenanthrene skeleton.

In one chemical synthetic strategy the lipophilic group-containing side chain is a C₁₂, C₁₄, C₁₆ or C₁₈ acyl group, for example a tetradecanoyl group, acylating an amino group present in the side chain of a residue of the backbone of the agonist.

As stated, the modification of agonists for use in accordance to provide improved serum binding characteristics is a strategy which may be applied in general, and particularly in the case of the specific agonists listed above. Thus suitable modified agonists include [N—(N′-tetradecanoyl)-gammagluatamoyl-Lys13,Ala30]PP2-36 and [Glu10,N—(N′-hexadecanoyl)-gammagluatamoyl-Lys13,Leu17,Thr30]PP2-36 and conservatively substituted analogues thereof.

GAG Binding

As in the case of lipophilic serum binding motifs discussed above, the agonists with which this invention are concerned may be modified by incorporation of the GAG binding motif as, or as part of, a side chain to the backbone of the agonist. Known GAG-binding motifs for incorporation in this way include the amino acid sequences XBBXBX and/or XBBBXXBX, wherein B is a basic amino acid residue and X is any amino acid residue. A plurality, for example three, of such sequences may be incorporated in a concatameric (straight chain) or dendrimeric (branched chain) fashion. Specific concatameric GAG motifs include Ala-Arg-Arg-Arg-Ala-Ala-Arg-Ala-Ala-Arg-Arg-Arg-Ala-Ala-Arg-Ala, and Ala-Arg-Arg-Arg-Ala-Ala-Arg-Ala-Ala-Arg-Arg-Arg-Ala-Ala-Arg-Ala-Ala-Arg-Arg-Arg-Ala-Ala-Arg-Ala (both of which may, for example be coupled through an amide bond formed between the C-terminus of the concatameric GAG-binding motif and an amino group in the side chain of a backbone amino acid of the agonist, such as the epsilon amino group of Lys13 in the agonist [Lys13,Ala30]PP2-36 or [Glu10,Lys13,Leu17,Thr30]PP2-36.

Instead of being attached to the agonist as, or as part of a side chain to a backbone residue, the GAG motif may be covalently linked to the C- or (preferably) N-terminus of the agonist, either directly or via a linker radical. Here also the GAG-binding motif may comprise the amino acid sequence XBBXBX and/or XBBBXXBX, wherein B is a basic amino acid residue and X is any amino acid residue, for example the sequence [XBBBXXBX]ₙ where n is 1 to 5, B is a basic amino acid residue and X is any amino acid residue. Such concatameric repeats tend to form alpha helices when they bind to GAG's, and consequently when fused to the C-terminal hexapeptide/last alpha helical turn, can stabilise that turn and thereby present the combined structure in an optimal way for Y4 receptor recognition. Specific examples of agonists of this type are [XBBBXXBX-XBBBXXBX]PP or [XBBBXXBX-XBBBXXBX-XBBBXXBX]PP, wherein B is a basic amino acid residue and X is any amino acid residue, particularly Ala-Arg-Arg-Arg-Ala-Ala-Arg-Ala-Ala-Arg-Arg-Arg-Ala-Ala-Arg-Ala-Ala-Arg-Arg-Arg-Ala-Ala-Arg-Ala-[Ala30]PP2-36.

The Y4 selective agonists with which the present invention is concerned are useful, inter alia, in indications for which prolonged exposure is desirable. For such indications in particular, the agonists preferably comprise a glycosamino glycan (GAG) binding motif as discussed above. Such motifs ensure that the agonists bind to GAGs in the extracellular matrix, and thereby ensures prolonged local exposure of the Y4 receptors in that tissue. Growth factors, chemokines etc bind to GAGs through patches of basic amino acids, which interact with the acidic sugars of the GAGs. These positively charged epitopes on the growth factors are usually composed of side chains from basic residues, which are not necessarily located consecutively in sequence but are often presented in close proximity by a secondary structural element such as an a-helix or a turn or by the overall three dimensional structure of the protein. Certain GAG-binding, linear sequences, discussed above, have been described, for example XBBXBX and XBBBXXBX where B represents a basic residue (Hileman et al. Bioassays 1998, 20: 156-67). These segments have been shown by circular dichroism to form α-helices upon binding to GAGs. If such sequences are placed for example in a concatameric or dendrimeric construct where for example three such sequences are presented—for example each as a ARRRAARA sequence—the resulting 24-mer peptide—for example ARRRAARA-ARRRAARA-ARRRAARA—ensures a retention in the extracellular matrix similar to high molecular weight polylysine, i.e. it is not washed out during a 4 hour perfusion period (Sakharov et al. FEBS Lett 2003, 27: 6-10).

Thus Growth factors and chemokines are naturally constructed with two types of binding motifs: one binding motif for the receptor through which signal transduction is achieved and one binding motif for GAG's through which attachment and long-lasting local activity is achieved. Peptides such as PYY and NPY are neuropeptides and hormones, which are rather rapidly washed out of the tissue and are not optimized for long-lasting local activity. By attaching a GAG-binding motif to a Y4 selective agonist according to the present invention—a bi-functional molecule similar to the growth factors and chemokines is constructed having both a receptor binding epitope in the PP-fold peptide part and a GAG-binding motif. An example of such an agonist is [N-{(Ala-Arg-Arg-Arg-Ala-Ala-Ala-Arg-Ala)3}-Lys13,Ala30]PP2-36.

### PEGylation

In PEGylation, a polyalkyleneoxide radical or radicals, is/are covalently coupled to peptidic or proteinaceous drugs to improve effective half life in the body following administration. The term derives from the preferred polyalkyleneoxide used in such processes, namely that derived from ethylene glycol—polyethyleneglycol, or “PEG”.

A suitable PEG radical may be attached to the agonist by any convenient chemistry, for example via a backbone amino acid residue of the agonist. For instance, for a molecule like e.g. PEG, a frequently used attachment group is the epsilon-amino group of lysine or the N-terminal amino group. Other attachment groups include a free carboxylic acid group (e.g. that of the C-terminal amino acid residue or of an aspartic acid or glutamic acid residue), suitably activated carbonyl groups, mercapto groups (e.g. that of a cysteine residue), aromatic acid residues (e.g. Phe, Tyr, Trp), hydroxy groups (e.g. that of Ser, Thr or OH-Lys), guanidine (e.g. Arg), imidazole (e.g. His), and oxidized carbohydrate moieties.

When the agonist is PEGylated it usually comprises from 1 to 5 polyethylene glycol (PEG) molecules such as, e.g. 1, 2 or 3 PEG molecules. Each PEG molecule may have a molecular weight of from about 5 kDa (kiloDalton) to about 100 kDa, such as a molecular weight of from about 10 kDa to about 40 kDa, e.g., about 12 kDa or preferably no more than about 20 kDa. In a particular embodiment of the invention, PEG 40 kDa (otherwise designated PEG40000) is the PEGylating agent.

Suitable PEG molecules are available from Shearwater Polymers, Inc. and Enzon, Inc. and may be selected from SS-PEG, NPC-PEG, aldehyde-PEG, mPEG-SPA, mPEG-SCM, mPEG-BTC, SC-PEG, tresylated mPEG (U.S. Pat. No. 5,880,255), or oxycarbonyl-oxy-N-dicarboxylmide-PEG (U.S. Pat. No. 5,122,614).

Particular examples of PEGylated agonists of the invention are [N-PEG5000-Lys13,Ala30]PP2-36 and [Glu10,N-PEG5000-Lys13,Leu17,Thr30]PP2-36 and [N-PEG20000Lys13]PP2-36, [N-PEG2000Lys13]PP2-36 and [N-PEG40000Lys13]PP2-36.

### Serum Albumin, GAG and PEG

Whether the modification to the agonist is attachment of a group to facilitate serum binding, GAG binding or improved stability via PEGylation, the serum albumin binding motif or GAG binding motif, or PEG radical may be, or may form part of, a side chain of a backbone carbon of the agonist corresponding to any of the following positions 1, 3, 6, 7, 10, 11, 12, 13, 15, 16, 18, 19, 21, 22, 23, 25, 26, 28, 29, and 32, although in the case of peptides [Glu10]PP₂₋₃₆ and [Glu10,Leu17,Thr30]PP₂₋₃₆ position 10 is not available.

### Conjugation to Larger Biomolecules

The selective Y4 receptor agonists may be used as fusion proteins where they are linked for example to albumin or another protein or carrier molecule which provides beneficial pharmacokinetic or other types of properties such as for example decreased renal elimination. There are multiple chemical modifications and linkers which can be used for such a covalent attachment as known in the art, just as there are multiple proteins or carriers which can be used. Especially covalent attachment of the selective Y4 peptide agonist to albumin is preferred and at one of the positions in the PP-fold structure, which have been pointed out elsewhere herein in relation to modifications with the various motifs. Such fusion proteins can be produced through various semi-synthetic techniques where the peptide may be made through peptide synthesis as described herein and the biomolecule through recombinant technology. The fusion protein may also be made enteriely as a recombinant molecule expressed for example as a precursor molecule extended by a Gly-Lys-Arg sequence, which when expressed as a secretory protein in eukaryotic cells will be cleaved by biosynthetic enzymes and the Gly turned into the carboxyamide on the C-terminal Tyr residue of the C-terminal Y4 receptor recognition sequence.

### Helix Inducing Peptides

Acylation of the N-terminus of the agonists with which the invention is concerned has been mentioned as a means of stabilising the agonist against the action of aminopeptidases. Another stabilising modification involves the covalent attachment of a stabilizing peptide sequence of 4-20 amino acid residues covalently at the N- and/or the C-terminus, preferably the N-terminus. The amino acid residues in such a peptide are selected from the group consisting of Ala, Leu, Ser, Thr, Tyr, Asn, Gln, Asp, Glu, Lys, Arg, His, Met and the like. In an interesting embodiment the N-terminal peptide attachment comprises 4, 5 or 6 Lys residues, for example Lys-Lys-Lys-Lys-Lys-Lys-[Ala30]PP2-36 These can be linked at the N-terminus of the PP-fold peptide agonist. A general description of such stabilizing peptide extensions is given in WO 99/46283 (Zealand Pharmaceuticals), which is hereby incorporated by reference.

The receptor agonists with which the invention is concerned may be prepared by well-known methods such as, e.g., a synthetic, semisynthetic and/or recombinant method. The methods include standard peptide preparation techniques such as, e.g., solution synthesis, and solid-phase synthesis. Based on textbook and general knowledge within the field, a person skilled in the art knows how to proceed in order to obtain the agonists and derivatives or modifications thereof.

### Utilities

In accordance with the invention, it has been found that selective Y4 receptor agonists (for example PP2-36) can induce increased cell proliferation within the crypts of Lieberkuhn in the small intestinal epithelium. This observation identifies a mechanism which may at least partially underlie the bowel function benefits of the selective Y4 receptor agonists. Agents which increase the epithelial mass or surface area of the bowel are indicated for use in preventing or treating loss of bowel function (by restoring or maintaining barrier function and overall intestinal integrity, preventing infection, diarrhea and sepsis). Thus the selective Y4 receptor agonists may be used, for example, to prevent or treat ulcerations that occur in intestinal mucositis, ulcerative colitis and Crohns disease. Stimulation of the epithelium is also a useful treatment following intestinal resection and in cases of short bowel syndrome, where the consequences of increased proliferation are an increased differentiated cell population capable of improving nutrition digestion and absorption. The increased proliferation mechanism also suggests use of the selective Y4 receptor agonists to prevent or treat intestinal reperfusion injury.

In one particular context, the Y4 receptor agonist used according to the invention is capable of alleviating damage to bowel function caused by radiation therapy, radiation exposure, cytotoxic chemotherapy, inflammation or ischemia-reperfusion of intestinal mucosa. In the case of damage characterized by mucosal cell loss, it appears to do so by encouraging restoration of intestinal cells. Hence the selective Y4 agonist may be administered prior and/or concurrently with the cytotoxic insult and/or after damage to mucosal function has occurred. In another particular context, treatment according to the invention alleviates intestinal mucositis (inflammation and ulceration of the mucous membranes lining the digestive tract), and the abdominal cramping and diarrhea associated with that condition. In yet another context the selective Y4 receptor agonist may be used for treatment of intestinal ischemia/reperfusion injury.

Because diarrhea is a main symptom of damage to bowel function caused by radiation therapy, radiation exposure, cytotoxic chemotherapy, inflammation or ischemia-reperfusion of intestinal mucosa a Y4 agonist used according to the present invention may be administered in combination with other agents known for use in treatment of diarrhea generally. Such agents include: Loperamide, Octreotide, Atropine, Tincure of opium, Diphenoxylate, Psyllium, Methylcellulose, Pectin, Activated charcoal, Probiotics (e.g. Lactobacillus acidophilus), Racecadotril (acetorphan), Glutamine, Celecoxib, Antibiotics, Kampo, Oral alkalinizing agents, Thalidomide, GLP-2 agonists, Y2 receptor agonists, 5-HT1, 5-HT2 and/or 5-HT7 receptor ligands not displaying 5-HT4 binding affinity, LPA2 receptor agonist inhibitors of CFTR, Selective antagonists of A2B adenosine receptors, Inhibitors of tryptophan hydroxylase (TPH), Compounds able of potentiate opioid receptor function, Derivatives containing an hydrogen sulfide (H2S)-releasing moiety, Bombesin 2 (BB2) receptor antagonists, Prokineticin 2 receptor (PK2) antagonists, Prokineticin 1 receptor (PK1) antagonists, Serotonin reuptake inhibitors (SSRI's), Selective vascular endothelial growth factor (VEGF) receptor tyrosine kinase inhibitory, Modulators of vanilloid VR1 receptors, 5HT4 receptor ligands, Delta opioid receptor modulators, Potassium channel regulators, Phospholipase inhibitors, Clonidine derivatives, Salts of tegaserod, Analogs of 7,8-saturated-4,5-epoxy-morphinaniu, Calcium receptor modulating agents, Phenylpropionamide compounds, Rifaximin, 5-chloro-6-(2-iminopyrrolidin-1-yl)methyl-2,4(1H,3H)-pyrimidinedione or analogous thereof, A pharmaceutical composition comprising a salt of hydrogen sulfide, Methylnaltrexone stereoisomer, Pantethine, Histidine or a derivative thereof, Fudosteine, Synthetic derivatives of the unnatural (+)-enantiomer of cannabidiol, N-(Cyclopropylmethyl)-azacycloalkanes and compositions containing them, Tramadol, Clotrimazole and related compounds, Indigestible oligosaccharides.

The selective Y4 receptor agonist can be administered by any route, including the enteral (e.g. rectal suppository administration, or oral administration—in which case the agonist may be coated with an enteric coating which allows it to pass through the stomach and disintegrates in the intestine), topical, or parenteral route. In a specific embodiment, the parenteral route is preferred and includes intravenous, intraarticular, intraperitoneal, subcutaneous, intramuscular, intrasternal injection and infusion as well as administration by the sublingual, transdermal, topical, transmucosal including nasal route, or by inhalation such as, e.g., pulmonary inhalation. Subcutaneous and/or nasal administration, and/or administration via a rectal suppository and/or administration of an oral enteric coated dose are all useable routes.

The selective Y4 receptor agonist can be administered as such, dispersed in a suitable vehicle, or in the form of a suitable pharmaceutical or cosmetic composition comprising the specific compound together with one or more physiologically or pharmaceutically acceptable excipients. A composition suitable for a specific administration route is easily determined by a medical practitioner for each patient individually. Various pharmaceutically acceptable carriers and their formulation are described in standard formulation treatises, e.g., Remington's Pharmaceutical Sciences by E. W. Martin.

The pharmaceutical composition comprising a compound according to the invention may be in the form of a solid, semi-solid or fluid composition. For parenteral use, the composition is normally in the form of a fluid composition or in the form of a semi-solid or solid form for implantation.

Fluid compositions, which are sterile solutions or dispersions can utilized by for example intravenous, intramuscular, intrathecal, epidural, intraperitoneal or subcutaneous injection of infusion. The compounds may also be prepared as a sterile solid composition, which may be dissolved or dispersed before or at the time of administration using e.g. sterile water, saline or other appropriate sterile injectable medium.

The fluid form of the composition may be a solution, an emulsion including nano-emulsions, a suspension, a dispersion, a liposomal composition, a mixture, a spray, or a aerosol (the two latter types are especially relevant for nasal administration).

Suitable mediums for solutions or dispersions are normally based on water or pharmaceutically acceptable solvents e.g. like an oil (e.g. sesame or peanut oil) or an organic solvent like e.g. propanol or isopropanol. A composition according to the invention may comprise further pharmaceutically acceptable excipients such as, e.g., pH adjusting agents, osmotically active agents e.g. in order to adjust the isotonicity of the composition to physiologically acceptable levels, viscosity adjusting agents, suspending agents, emulsifiers, stabilizers, preservatives, antioxidants etc. A preferred medium is water.

Compositions for nasal administration may also contain suitable non-irritating vehicles such as, e.g., polyethylene glycols, glycofurol, etc. as well as absorption enhancers well known by a person skilled in the art (e.g. with reference to Remington's Pharmaceutical Science)

For parenteral administration, in one embodiment the receptor agonists can be formulated generally by mixing it at the desired degree of purity, in a unit dosage injectable form (solution, suspension, or emulsion), with a pharmaceutically acceptable excipient or carrier, i.e., one that is non-toxic to recipients at the dosages and concentrations employed and is compatible with other ingredients of the composition.

The compositions may also be designed to controlled or prolonged delivery of the receptor agonist after administration in order to obtain a less frequent administration regimen. Normally a dosage regimen including 1-2 daily administrations is considered suitable, but within the scope of the present invention is also included other administration regimens such as, e.g., more frequent and less frequent. In order to achieve a prolonged delivery of the receptor agonist, a suitable vehicle including e.g. lipids or oils may be employed in order to form a depot at the administration site from which the receptor agonist is slowly released into the circulatory system, or an implant may be used. Suitable compositions in this respect include liposomes and biodegradable particles into which the receptor agonist has been incorporated.

In those situations where solid compositions are required, the solid composition may be in the form of tablets such as, e.g. conventional tablets, effervescent tablets, coated tablets, melt tablets or sublingual tablets, pellets, powders, granules, granulates, particulate material, solid dispersions or solid solutions.

A semi-solid form of the composition may be a chewing gum, an ointment, a cream, a liniment, a paste, a gel or a hydrogel.

Other suitable dosages forms of the pharmaceutical compositions according to the invention may be vagitories, suppositories, plasters, patches, tablets, capsules, sachets, troches, devices etc.

The dosage form may be designed to release the compound freely or in a controlled manner e.g. with respect to tablets by suitable coatings.

The content of the Y4 agonist of the invention in a pharmaceutical composition of the invention is e.g. from about 0.1 to about 100% w/w of the pharmaceutical composition, but optimum dosages will be determined by clinical trial, as is required by law in the art.

The following Examples illustrate aspects of the invention:

### 1. In Vitro Assays to Determine Peptide Potency

### Human Y2 Receptor Potency Assay

Potency of the test compounds on the human Y2 receptor is determined by performing dose-response experiments in COS-7 cells transiently transfected with the human Y2 receptor cDNA as well as a promiscuous G protein, Gqi5 which ensures that the Y2 receptor couples through a Gq pathway leading to an increase in inositol phosphate turnover.

Phosphatidylinositol turnover—One day after transfection COS-7 cells are incubated for 24 hours with 5 μCi of [3H]-myo-inositol (Amersham, PT6-271) in 1 ml medium supplemented with 10% fetal calf serum, 2 mM glutamine and 0.01 mg/ml gentamicin per well. Cells are washed twice in buffer, 20 mM HEPES, pH 7.4, supplemented with 140 mM NaCl, 5 mM KCl, 1 mM MgSO4, 1 mM CaCl2, 10 mM glucose, 0.05% (w/v) bovine serum; and are incubated in 0.5 ml buffer supplemented with 10 mM LiCl at 37 C for 30 min. After stimulation with various concentrations of peptide for 45 min at 37 C, cells are extracted with 10% ice-cold perchloric acid followed by incubation on ice for 30 min. The resulting supernatants are neutralized with KOH in HEPES buffer, and the generated [3H]-inositol phosphate are purified on Bio-Rad AG 1-X8 anion-exchange resin and counted in a beta counter. Determinations are made in duplicates. EC50 values were calculated using a standard pharmacological data handling software, Prism 3.0 (graphPad Sofware, San Diego, USA).

### Human Y4 Receptor Potency Assay

Protocol as for the Y2 potency assay, except that COS-7 cells are transiently transfected with human Y4 receptor cDNA.

### Human Y1 Receptor Potency Assay

Protocol as for the Y2 potency assay, except that COS-7 cells are transiently transfected with human Y1 receptor cDNA.

### Human Y5 Receptor Potency Assay

Protocol as for the Y2 potency assay, except that COS-7 cells are transiently transfected with human Y5 receptor cDNA.

The Y4 agonists SEQ ID Nos 3-35 herein all have potencies at least 50 fold (actually at least 200) fold greater potency at the Y4 receptor than at the Y1 receptor, and at least 1000 fold greater potency at the Y4 receptor than at the Y2 receptor when tested in the above assays

### 2. Effect of a Selective Y4 Receptor Agonist on Intestinal Mucosal Cell Loss

The following experiment shows that treatment of mice with PP[2-36] (SEQ ID No:4) s.c. increases the number of surviving small intestinal crypts following irradiation exposure.

### Methods:

Twenty four 8-10 week old C57/B6 male mice randomized into three groups of each eight animals. Animals received PP[2-36] (0.1 mg/kg) s.c. twice daily for three days either prior or post radiation. Controls received vehicle both prior and post radiation.

Group 1 (pretreatment): PP[2-36] (0.1 mg/kg) twice daily on day −3, −2, −1; radiation on day 0; vehicle on day 0, +1, +2, +3. Group 2 (post treatment): Vehicle twice daily on day −3, −2, −1; radiation on day 0; PP[2-36] (0.1 mg/kg twice daily on day 0, +1, +2, +3. Group 3 (control): Vehicle twice daily on day −3, −2, −1; radiation on day 0; vehicle twice daily on day 0, +1, +2, +3. All animals were irradiated (day 0) to a single dose of 13Gy total body X-irradiation. Irradiation was performed using a Pantak HF320 X-ray set (Agfa NDT Ltd, Reading, UK). The machine was operated at 300 kV, 10 mA. The X-ray tube was fitted with additional filtration to give a radiation quality of 2.3 mm Cu half-value layer (HVL). Mice were restrained in a jig, positioned at a distance of 700 mm from the focus of the X-ray tube. Irradiation was delivered at a dose rate of 75.5 cGy/min. Four days post irradiation insult, the mice were sacrificed by cervical dislocation. The small intestine was removed, fixed in Carnoy's fixative, paraffin-embedded, sectioned and H&E stained. For each animal ten intestinal circumferences were analyzed—a circumference is equivalent to a given length of intestine and therefore a convenient baseline unit of length. The number of surviving crypts per circumference was scored and the average per group determined. Only crypts containing 10 or more strongly H&E stained cells (excluding Paneth cells) and only intact circumferences not containing Peyers patches were scored (Peyers patches influence both the number of crypts in a normal circumference and the ability of a crypt to survive insult).

### Results:

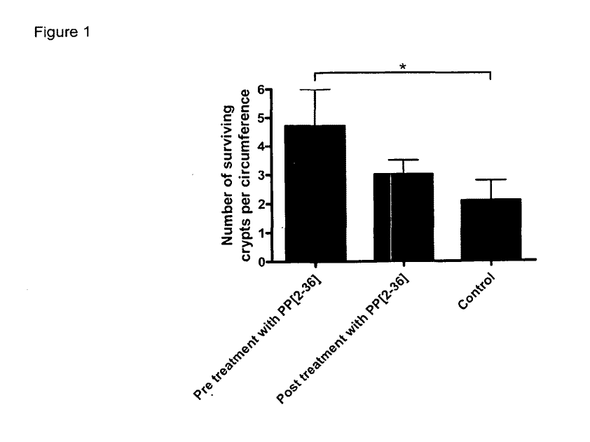

Treatment of animals prior radiation with PP[2-36] had a beneficial effect on crypt survival and/or regeneration compared to vehicle control. The results are summarised in FIG. 1 which shows the mean number of surviving crypts per intestinal circumference in each treatment group following irradiation. Pretreatment of mice with 0.1 mg/kg PP[2-36] s.c twice daily for three days prior irradiation increase the number of surviving intestine crypts by two fold compared to vehicle control when analyzed four days following irradiation (significant; p<0.05). Treatment of mice with 0.1 mg/kg PP[2-36] s.c. twice daily for four days post irradiation increase number surviving crypts by 47% compared to vehicle control when analyzed four days following irradiation (non-significant; p>0.05). Data are expressed as means±SD (Tukey-Kramer HSD; * p<0.05).

### 3. Effect of a Selective Y4 Receptor Agonist on Intestinal Mucosal Cell Proliferation

It was hypothesised that the beneficial effects on crypt survival and regeneration observed in section 2 above may be due, at least in part, to increased proliferation of cells of the crypt due to treatment with the selective Y4 receptor agonist agent. To test that hypothesis, the following experiment was performed.

### Methods:

3 Groups of 8 male 8-10 weeks old C57/B6 mice were treated with group 1: Vehicle, group 2:PP(2-36) 0,1 mg/kg single injection s.c., group 3: PP(2-36) 1,0 mg/kg single injection s.c. Animals were euthanized 12 hours following the single injection of either dose. Vehicle was used as control. Prior to euthanasia all animals were given BrdU (Bromodeoxyuridine) i.p.—a marker for cell proliferation. The small intestine and colon were then removed and fixed in Carnoy's solution. From the Carnoy's fixed small intestinal material, paraffin blocks were generated and sectioned. Slides were immunolabelled to reveal the BrdU incorporation and analyzed on a cell positional basis (i.e. location of the individual cell in the hierarchy of cells in the crypt) to identify any induced proliferative changes (BrdU incorporation and mitotic counts). Fifty half crypts per mouse were scored on a cell positional basis, generating 400 frequency scores per group of 8 animals from which the means were generated and effects analyzed.

### Results:

The results are summarised in FIGS. 2a and 2b. Twelve hours following a dose of PP(2-36) there was a statistical significant increase in the level of proliferation in the small crypts. 0,1 mg/kg increased proliferation at cell positions 6-13 (stem and early transit amplifying cell region). A stem cell is defined as an undifferentiated cell capable of proliferation, self maintenance, production of a large number of differentiated functional progeny, regeneration of the tissue after injury and a flexibility in the use of these options. Stem cell daughter cells do not express all these capabilities, but have the potential to do so under extreme circumstances, they are named potential stem cells and together with the stem cells they are named clonogenic cells. A cell that is not fulfilling any clonogenic function and is simply committed to terminal differentiation is termed a transit amplifying cell—a relatively short lived cell that ultimately differentiates and provides a function upon the villus, before being shed into the gut lumen. When the dose was increased to 1 mg/kg stimulation was evident throughout the proliferative zone.

In summary, these results show that treatment of mice with the selective Y4 receptor agonist PP(2-36) (SEQ ID No:4) administered as a single subcutaneous injection increases cell proliferation within the crypt.

1-7. (canceled)

8. A method of prevention and/or treatment of damage to bowel function caused by radiation therapy, radiation exposure, cytotoxic chemotherapy, inflammation or ischemia-reperfusion of intestinal mucosa in a subject in need thereof comprising administering to the subject a therapeutically effective amount of a Y4 receptor agonist, wherein the Y4 receptor agonist has
(a) at least 50 fold greater potency at the Y4 receptor than at the Y1 receptor, and
(b) at least 1000 fold greater potency at the Y4 receptor than at the Y2 receptor.

9. The method of claim 8 wherein the Y4 receptor agonist has at least 100 fold greater potency at the Y4 receptor than at the Y1 receptor.

10. The method of claim 8 wherein the Y4 receptor agonist has at least 200 fold greater potency at the Y4 receptor than at the Y1 receptor.

11. The method of claim 10, wherein the Y4 receptor agonist is selected from SEQ ID NOS:3-35.

12. The method of claim 8 wherein the damage to bowel function is caused by inflammatory bowel disease.

13. The method of claim 8, further comprising administering at least one other diarrhea treatment agent to the subject.

14. The method of claim 12 wherein the inflammatory bowel disease is ulcerative colitis.

15. The method of claim 12 wherein the inflammatory bowel disease is Crohn's disease.


SEQUENCE LISTING (44 sequences)

SEQ ID NO: 1 - protein, 36 aa, Homo sapiens
  Amidation (36)..(36)
YPSKPDNPGEDAPAEDMARYYSALRHYINLITRQRY

SEQ ID NO: 2 - protein, 36 aa, Homo sapiens
  Amidation (36)..(36)
YPIKPEAPGEDASPEELNRYYASLRHYLNLVTRQRY

SEQ ID NO: 3 - protein, 36 aa, Homo sapiens
  Amidation (36)..(36)
APLEPVYPGDNATPEQMAQYAADLRRYINMLTRPRY

SEQ ID NO: 4 - protein, 35 aa, Homo sapiens
  Amidation (35)..(35)
PLEPVYPGDNATPEQMAQYAADLRRYINMLTRPRY

SEQ ID NO: 5 - protein, 34 aa, Homo sapiens
  Amidation (34)..(34)
LEPVYPGDNATPEQMAQYAADLRRYINMLTRPRY

SEQ ID NO: 6 - protein, 35 aa, Artificial Sequence
  Modified human Pancreatic Polypeptide
PLEPVYPGDNATPEQMAQYAADLRRYINALTRPRY

SEQ ID NO: 7 - protein, 36 aa, Artificial Sequence
  Modified human Pancreatic Polypeptide
APLEPVYPGDNATPEQMAQYAADLRRYINALTRPRY

SEQ ID NO: 8 - protein, 34 aa, Artificial Sequence
  Modified human Pancreatic Polypeptide
LEPVYPGDNATPEQMAQYAADLRRYINALTRPRY

SEQ ID NO: 9 - protein, 35 aa, Artificial Sequence
  Modified human Pancreatic Polypeptide
PLEPVYPGDNATPEQMAQYAADLRRYINTLTRPRY

SEQ ID NO: 10 - protein, 36 aa, Artificial Sequence
  Modified human Pancreatic Polypeptide
APLEPVYPGDNATPEQMAQYAADLRRYINTLTRPRY

SEQ ID NO: 11 - protein, 34 aa, Artificial Sequence
  Modified human Pancreatic Polypeptide
LEPVYPGDNATPEQMAQYAADLRRYINTLTRPRY

SEQ ID NO: 12 - protein, 35 aa, Artificial Sequence
  Modified human Pancreatic Polypeptide
PLEPVYPGDNATPEQMAQYAADLRRYINNLTRPRY

SEQ ID NO: 13 - protein, 36 aa, Artificial Sequence
  Modified human Pancreatic Polypeptide
APLEPVYPGDNATPEQMAQYAADLRRYINNLTRPRY

SEQ ID NO: 14 - protein, 34 aa, Artificial Sequence
  Modified human Pancreatic Polypeptide
LEPVYPGDNATPEQMAQYAADLRRYINNLTRPRY

SEQ ID NO: 15 - protein, 35 aa, Artificial Sequence
  Modified human Pancreatic Polypeptide
PLEPVYPGDNATPEQMAQYAADLRRYINQLTRPRY

SEQ ID NO: 16 - protein, 36 aa, Artificial Sequence
  Modified human Pancreatic Polypeptide
APLEPVYPGDNATPEQMAQYAADLRRYINQLTRPRY

SEQ ID NO: 17 - protein, 34 aa, Artificial Sequence
  Modified human Pancreatic Polypeptide
LEPVYPGDNATPEQMAQYAADLRRYINQLTRPRY

SEQ ID NO: 18 - protein, 35 aa, Artificial Sequence
  Modified human Pancreatic Polypeptide
PLEPVYPGENATPEQMAQYAADLRRYINMLTRPRY

SEQ ID NO: 19 - protein, 36 aa, Artificial Sequence
  Modified human Pancreatic Polypeptide
APLEPVYPGENATPEQMAQYAADLRRYINMLTRPRY

SEQ ID NO: 20 - protein, 34 aa, Artificial Sequence
  Modified human Pancreatic Polypeptide
LEPVYPGENATPEQMAQYAADLRRYINMLTRPRY

SEQ ID NO: 21 - protein, 35 aa, Artificial Sequence
  Modified human Pancreatic Polypeptide
PLEPVYPGENATPEQLAQYAADLRRYINTLTRPRY

SEQ ID NO: 22 - protein, 36 aa, Artificial Sequence
  Modified human Pancreatic Polypeptide
APLEPVYPGENATPEQLAQYAADLRRYINTLTRPRY

SEQ ID NO: 23 - protein, 34 aa, Artificial Sequence
  Modified human Pancreatic Polypeptide
LEPVYPGENATPEQLAQYAADLRRYINTLTRPRY

SEQ ID NO: 24 - protein, 35 aa, Artificial Sequence
  Modified human Pancreatic Polypeptide
PLEPVYPGDNATPEQXAQYAADLRRYINXLTRPRY

SEQ ID NO: 25 - protein, 36 aa, Artificial Sequence
  Modified human Pancreatic Polypeptide
APLEPVYPGDNATPEQXAQYAADLRRYINXLTRPRY

SEQ ID NO: 26 - protein, 34 aa, Artificial Sequence
  Modified human Pancreatic Polypeptide
LEPVYPGDNATPEQXAQYAADLRRYINXLTRPRY

SEQ ID NO: 27 - protein, 35 aa, Artificial Sequence
  Modified human Pancreatic Polypeptide
PLEPVYPGENATPEQXAQYAADLRRYINXLTRPRY

SEQ ID NO: 28 - protein, 36 aa, Artificial Sequence
  Modified human Pancreatic Polypeptide
APLEPVYPGENATPEQXAQYAADLRRYINXLTRPRY

SEQ ID NO: 29 - protein, 34 aa, Artificial Sequence
  Modified human Pancreatic Polypeptide
LEPVYPGENATPEQXAQYAADLRRYINXLTRPRY

SEQ ID NO: 30 - protein, 35 aa, Artificial Sequence
  Modified human Pancreatic Polypeptide
PLEPVYPGDNATPEQLAQYAADLRRYINTLTRPRY

SEQ ID NO: 31 - protein, 36 aa, Artificial Sequence
  Modified human Pancreatic Polypeptide
APLEPVYPGDNATPEQLAQYAADLRRYINTLTRPRY

SEQ ID NO: 32 - protein, 34 aa, Artificial Sequence
  Modified human Pancreatic Polypeptide
LEPVYPGDNATPEQLAQYAADLRRYINTLTRPRY

SEQ ID NO: 33 - protein, 35 aa, Artificial Sequence
  Modified human Pancreatic Polypeptide
PLEPVYPGDNATPEQLAQYAADLRRYINSLTRPRY

SEQ ID NO: 34 - protein, 36 aa, Artificial Sequence
  Modified human Pancreatic Polypeptide
APLEPVYPGDNATPEQLAQYAADLRRYINSLTRPRY

SEQ ID NO: 35 - protein, 34 aa, Artificial Sequence
  Modified human Pancreatic Polypeptide
LEPVYPGDNATPEQLAQYAADLRRYINSLTRPRY

SEQ ID NO: 36 - protein, 6 aa, Artificial Sequence
  GAG binding motif
XXXXXX

SEQ ID NO: 37 - protein, 8 aa, Artificial Sequence
  GAG binding motif
XXXXXXXX

SEQ ID NO: 38 - protein, 16 aa, Artificial Sequence
  GAG binding motif
ARRRAARAARRRAARA

SEQ ID NO: 39 - protein, 24 aa, Artificial Sequence
  GAG binding motif
ARRRAARAARRRAARAARRRAARA

SEQ ID NO: 40 - protein, 52 aa, Artificial Sequence
  modified human pancreatic polypeptide
XXXXXXXXXXXXXXXXAPLEPVYPGDNATPEQMAQYAADLRRYINMLTRPRY

SEQ ID NO: 41 - protein, 60 aa, Artificial Sequence
  modified human pancreatic polypeptide
XXXXXXXXXXXXXXXXXXXXXXXXAPLEPVYPGDNATPEQMAQYAADLRRYINMLTRPRY

SEQ ID NO: 42 - protein, 59 aa, Artificial Sequence
  modified human pancreatic polypeptide
ARRRAARAARRRAARAARRRAARAPLEPVYPGDNATPEQMAQYAADLRRYINALTRPRY

SEQ ID NO: 43 - protein, 8 aa, Artificial Sequence
  GAG binding motif
ARRRAARA

SEQ ID NO: 44 - protein, 62 aa, Artificial Sequence
  modified human pancreatic polypeptide
ARRRAAARAARRRAAARAARRRAAARAPLEPVYPGDNAKPEQMAQYAADLRRYINALTRPRY